SEQUENCE LISTING (3 sequences)

SEQ ID NO: 1 - protein, 134 aa, Homo sapiens
QPRPAFSAIRRNPPMGGNVVIFDTVITNQEEPYQNHSGRFVCTVPGYYYFVFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 135 aa, Homo sapiens
TQKIAFSATRTINVPLRRDQTIRFDHVITNMNNNYEPRSGKFTCKVPGLYTFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 3 - protein, 129 aa, Homo sapiens
KFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

(19) United States
(12) Patent Application Publication
(10) Pub. No.: US 2021/0405037 A1
(43) Pub. Date: Dec. 30, 2021

(54) Electrochemical Antibody-Based Biosensor

(71) Applicant: Northeastern University, Boston, MA (US)

(72) Inventor: Edgar D. GOLUCH, Somerville, MA (US)

(21) Appl. No.: 17/371,786

(22) Filed: Jul. 9, 2021

Related U.S. Application Data
(62) Division of application No. 16/480,079, filed on Jul. 23, 2019, now abandoned.
(60) Provisional application No. 62/450,073, filed on Jan. 25, 2017.

(51) Int. Cl. G01N 33/543; G01N 27/327; G01N 33/68

(52) U.S. Cl. CPC G01N 33/5438; G01N 33/6854; G01N 27/3277; G01N 27/3276

(57) ABSTRACT

Methods and sensors using antibody-based electrochemical detection of analytes including small molecules make use of the specific recognition of analyte-bound antibody by the complement system protein C1q. The antibody is immobilized to an electrode to which a potential is applied and the C1q protein is linked to a redox-active molecule, such that binding of C1q to the analyte-bound antibody brings the redox-active molecule in contact with the electrode, whereupon the analyte is detected by an increase in current.

### CROSS REFERENCE TO RELATED APPLICATIONS

This is a divisional application of U.S. application Ser. No. 16/480,079, filed 23 Jul. 2019, which is the U.S. national phase of PCT Application No. PCT/US2018/015329, filed 25 Jan. 2018. This application also claims the benefit of U.S. Provisional Application No. 62/450,073 filed 25 Jan. 2017 and entitled “Redox-Based Detection of Antibody Binding Events”. Each of the aforementioned applications is hereby incorporated by reference.

### BACKGROUND

Simple and rapid sensors for detection of small biological molecules are scarce. The most popular approach for detecting biological molecules, the enzyme-linked immunosorbent assay (ELISA), often cannot be used for sensing small molecules, or molecules not having at least two distinctly accessible epitopes. ELISA typically is carried out in the “sandwich” format, in which a target molecule (antigen) is first bound to an antibody that is immobilized on a surface, followed by identification of the binding event with a second antibody that attaches to a different location on the antigen. While many small biomolecules produce an antibody response, i.e., antibodies can be developed that bind to them, the physical size of these molecules is too small to permit attachment of two different antibodies to them. To work around this limitation, competition assays have been utilized in which a synthetic molecule is created that competes against the antigen for attachment to the immobilized antibody. Detection of the antigen then requires determining the amount of synthetic molecules that bind to the antibodies. This approach requires development of a new synthetic molecule for each antigen of interest.

A more sophisticated approach that avoids use of two antibodies instead employs label-free techniques that allow detection of binding of antigen onto antibodies immobilized on the sensor surface. However, low molecular weight molecules are often also too small to be detected by common label-free techniques such as Surface Plasmon Resonance (SPR) or Quartz Crystal Microbalance (QCM) sensors. Other label-free techniques such as mass spectrometry require extensive sample processing and large equipment.

There is a need to develop techniques that are designed for sensing low molecular weight molecules, and also rapid and convenient methods for detecting and quantifying biomolecules.

### SUMMARY

One aspect of the present technology is a kit for antibody-based electrochemical detection of an analyte. The kit includes an electrode having a detection surface; an antibody covalently linked to the detection surface; and a C1q polypeptide, or a fragment or variant thereof, covalently linked to a redox active molecule by a tether. The antibody is capable of specifically binding to the analyte, and the C1q polypeptide, or fragment or variant thereof, is capable of binding to a complex formed by binding of the analyte to the antibody and thereby contacting the redox active molecule with the detection surface.

Another aspect of the technology is a device for antibody-based electrochemical detection of an analyte. The device includes a working electrode having a detection surface; an antibody covalently linked to the detection surface; a reference electrode; and circuitry for applying a voltage between the working electrode and the reference electrode and measuring a current produced by a redox reaction at the detection surface. The antibody is capable of specifically binding to the analyte.

Yet another aspect of the technology is a method for detecting an analyte. The method includes the steps of: (a) providing the device described above, a sample suspected of containing the analyte, and a C1q polypeptide, or a fragment or variant thereof, covalently linked to a redox active molecule by a tether; (b) contacting the sample with the detection surface of the device, whereby the antibody binds to analyte in the sample to form an antibody-analyte complex; (c) contacting the C1q polypeptide, or fragment or variant thereof, with the antibody-analyte complex, whereby the redox active molecule contacts the detection surface; and (d) applying a voltage between the working electrode and the reference electrode of the device, whereby an electron transfer reaction of the redox active molecule is detected by the device as a current between the working electrode and the reference electrode. The C1q polypeptide, or fragment or variant thereof, is capable of binding to the analyte-antibody complex.

The technology is further summarized by the following list of embodiments.

1. A kit for antibody-based electrochemical detection of an analyte, the kit comprising:

an electrode having a detection surface;

an antibody covalently linked to the detection surface, wherein the antibody is capable of specifically binding to the analyte; and

a C1q polypeptide, or a fragment or variant thereof, covalently linked to a redox active molecule by a tether, wherein the C1q polypeptide, or fragment or variant thereof, is capable of binding to a complex formed by binding of the analyte to the antibody and thereby contacting the redox active molecule with the detection surface.

2. The kit of embodiment 1 comprising a C1q polypeptide variant, wherein the variant has at least 95% identity with the amino acid sequence of a mammalian C1q polypeptide.
3. The kit of embodiment 1 or embodiment 2, further comprising one or more additional antibodies covalently linked to said detection surface or to one or more additional detection surfaces on one or more additional electrodes, wherein each of said one or more additional antibodies is capable of specifically binding to a unique additional analyte.
4. The kit of embodiment 3, comprising two or more separate electrodes, each electrode comprising a detection surface to which is bound a unique antibody that specifically binds to a unique analyte.
5. The kit of any of the previous embodiments, wherein the redox active molecule has a half-wave potential in the range from about −0.4 volts to about 0.0 volts with respect to a Ag/AgCl reference electrode.
6. The kit of embodiment 5, wherein the redox active molecule is methylene blue.
7. The kit of any of the previous embodiments, wherein the analyte has a molecular weight of less than 10,000 Daltons.
8. The kit of embodiment 7, wherein the analyte has a molecular weight of less than 1,000 Daltons.
9. The kit of any of the previous embodiments, wherein the analyte is a biomolecule.
10. The kit of embodiment 9, wherein the biomolecule is a cytokine, a hormone, a peptide, a polypeptide, a nucleic acid, a sugar, or a polysaccharide.
11. The kit of embodiment 9, wherein the biomolecule is from a pathogen.
12. The kit of embodiment 9, wherein the biomolecule has a molecular weight of 10,000 Daltons or greater.
13. The kit of any of the previous embodiments, wherein the analyte is a bacterial toxin or a mycotoxin.
14. The kit of any of embodiments 1-12, wherein the analyte is a pharmaceutical agent.
15. A device for antibody-based electrochemical detection of an analyte, the device comprising:

a working electrode having a detection surface;

an antibody covalently linked to the detection surface, wherein the antibody is capable of specifically binding to the analyte;

a reference electrode; and

circuitry for applying a voltage between the working electrode and the reference electrode and measuring a current produced by a redox reaction at the detection surface.

16. The device of embodiment 15, further comprising one or more additional working electrodes, each additional working electrode comprising a detection surface to which is bound a unique antibody that specifically binds to a unique analyte.
17. A method for detecting an analyte, the method comprising the steps of:

(a) providing the device of embodiment 15; a sample suspected of containing the analyte; and a C1q polypeptide, or a fragment or variant thereof, covalently linked to a redox active molecule by a tether, wherein the C1q polypeptide, or fragment or variant thereof, is capable of binding to a complex formed by binding of the analyte to the antibody covalently linked to the detection surface of the device;

(b) contacting the sample with the detection surface of the device, whereby the antibody covalently linked to the detection surface binds to analyte in the sample to form an antibody-analyte complex;

(c) contacting the C1q polypeptide, or fragment or variant thereof, with the antibody-analyte complex, whereby the redox active molecule contacts the detection surface; and

(d) applying a voltage between the working electrode and the reference electrode of the device, whereby an electron transfer reaction of the redox active molecule is detected by the device as a current between the working electrode and the reference electrode.

18. The method of embodiment 17, wherein current is measured in step (c) in response to one or more square wave potentials.
19. The method of embodiment 17 or embodiment 18, further comprising determining a concentration of the analyte by applying a previously determined correlation between the current measured in step (d) and concentration of the analyte.
20. The method of any of embodiments 17-19, wherein the device further comprises one or more additional working electrodes, each additional working electrode comprising a detection surface to which is bound a unique antibody that specifically binds to a unique analyte, and two or more analytes are detected.

### BRIEF DESCRIPTION OF THE DRAWINGS

FIG. 1 shows a schematic representation of an electrochemical sensing method that utilizes C1q polypeptide coupled with a redox active molecule as a recognition element for binding to a complex between an antibody and an analyte.

FIG. 2 shows a schematic representation of a protein complex (left) formed in the complement system after IgG antibodies attach to a target antigen on a cell surface. To the right is shown a schematic diagram of a C1q-C1r-C1s protein complex.

FIG. 3 depicts structures of four mycotoxins as examples of small biological molecules that can be detected using the approach shown in FIG. 1. From top to bottom the mycotoxins are: aflatoxin B1, ochratoxin A, T-2 toxin, and satratoxin H.

FIG. 4 shows a diagram of a method of determining binding kinetics, such as for binding of C1q polypeptide to a surface-bound antibody, using SPR. When biomolecules attach to the surface of the sensor, the incidence angle shifts and incident light is reflected from the sensor surface onto the detector. The intensity of the light is proportional to the amount of material on the surface. The process is monitored in real time and binding kinetics are determined from the association and dissociation rates.

FIG. 5 shows a photomicrograph (right) of an embodiment of an electrochemical sensor that includes an integrated palladium reference electrode, and a photograph of the sensor on a chip (left). The sensor allows sub-micromolar sensitivity with sample volumes below 100 μL.

### DETAILED DESCRIPTION

The present technology provides antibody-based electrochemical sensors for detecting a wide variety of analytes, including both small molecules and macromolecules such as biomolecules. The technology utilizes a molecular interaction between an antibody and a C1q polypeptide, a component of the complement system, and makes it possible for antibody binding of an antigen to be detected electrochemically.

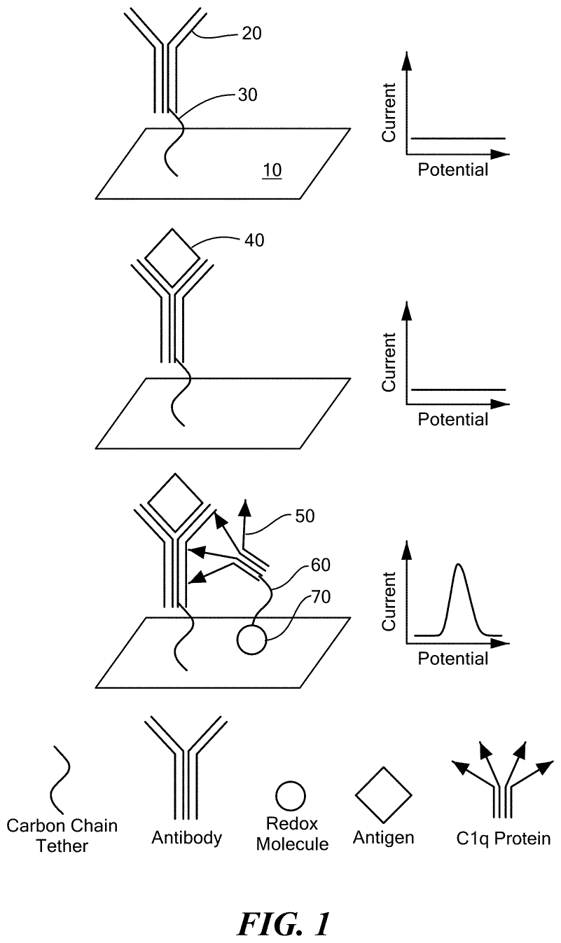

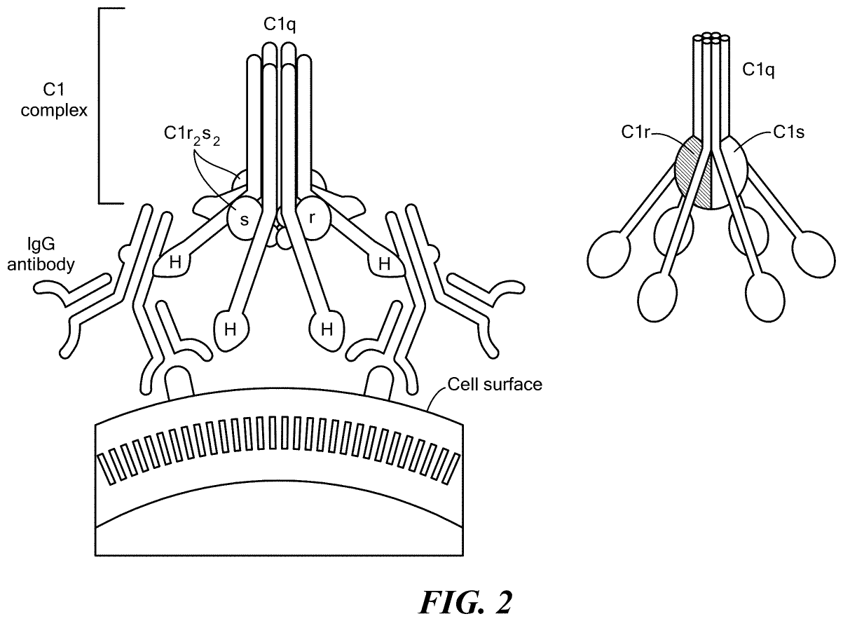

A schematic diagram of the process of detection using the sensor is shown in FIG. 1. Working electrode 10 of an electrochemical device has a detection surface (i.e., a surface of the electrode) to which antibody 20 is covalently or non-covalently attached. In FIG. 1 the antibody is attached via optional linker 30, which can be omitted. The antibody is selected for its specific binding of analyte 40, which triggers a conformational change in a constant region of the antibody, thereby allowing the binding of C1q polypeptide 50, which is covalently or non-covalently attached via tether 60 to redox active molecule 70. The binding of C1q to the antibody-analyte complex brings the redox active molecule into close proximity to the detection surface of the electrode. If a suitable voltage is applied between the working electrode and a reference electrode of the device, corresponding to the redox potential of the redox active molecule, a reaction occurs that produces a current between the electrodes, which is detected by the device as a signal that the analyte has been detected.

In order to practice the present technology, C1q protein is linked to a redox-active molecule, such that when it binds to an antibody attached to the surface of an electrode, a redox reaction takes place involving the redox active molecule at the electrode. The resulting electron transfer is manifested as an increase in current. In this manner, the electrochemical sensor allows voltammetric detection of antibody binding events. Redox active molecules for use in the present technology can be any molecule capable of electrochemical detection by oxidation or reduction at an electrode, and capable of covalent linkage to a tether that joins the redox active molecule to C1q. Preferably, the redox active molecule has a half-wave potential in the range from about −0.4 volts to about 0.0 volts with respect to a Ag/AgCl reference electrode, so that its signal does not overlap with that typically observed for biomolecules likely to be present in the sample. An example of a suitable redox active molecule is methylene blue.

The redox-active molecule is linked to C1q by a tether. The length of the tether can be optimized such that it is sufficient to allow the redox molecule to reach the electrode surface while not being so great as would make the molecule less likely to touch the electrode surface because of the additional freedom of movement imparted by the excess length. Optionally, the antibody molecules can be attached to the electrode surface by a second tether. Different lengths of the second tether also can be tested to select an optimum length that maximizes binding of C1q to a given antibody.

In the course of a humoral immune response, antibodies bound to antigens are specifically recognized by the complement system, a group of 20 proteins that help antibodies and macrophages clear pathogens from the body (Janeway, C A et al., 2001). All IgG (except IgG₄) and IgM antibodies undergo a conformational change in their Fc region when they bind to an antigen. Thus, antibodies for use in the present technology can be any form of IgG except IgG₄, or any IgM. This change is recognized by C1q, a large hexameric protein complex of the complement system. Each monomer of the C1q hexamer is made of three polypeptide chains, each of which is encoded by a separate gene. Altogether, a C1q complex contains 18 polypeptide chains, 6 A chains, 6 B chains, and 6 C chains. The A, B, and C chains are encoded by different genes and the polypeptides all share the same topology, including a globular N-terminal domain, a collagen-like central region, and a conserved C-terminal region.

The methods and sensors described herein utilize binding of C1q to antigen-bound antibodies to produce a new class of electrochemical sensors for detecting a binding event between an antibody and an analyte. The analyte can be a small molecule, for example. A “small molecule” as used herein can be a molecule having a molecular weight of less than about 2000 Da, or less than about 1800 Da, or less than about 1500 Da, or less than about 1200 Da, or less than about 1000 Da, or less than about 800 Da, or less than about 500 Da. Small molecule analytes suitable for detection and/or quantification using the present technology include metabolites, sugars, antibiotics, toxins, pharmaceutical agents, nutraceutical agents, components of food products, and plant-derived or fungus-derived compounds. While the technology has certain advantages (i.e., requiring only a single specifically binding antibody, rather than two that do not sterically interfere) over other technologies for detecting and/or quantifying small molecules, it also has advantages for large molecules, such as biomolecules. Such advantages include rapid readout of data and use of inexpensive and portable equipment. Thus, the methods and sensors of the present technology also can be used to detect and/or quantify cytokines, hormones, peptides, proteins, nucleic acids, and polysaccharides.

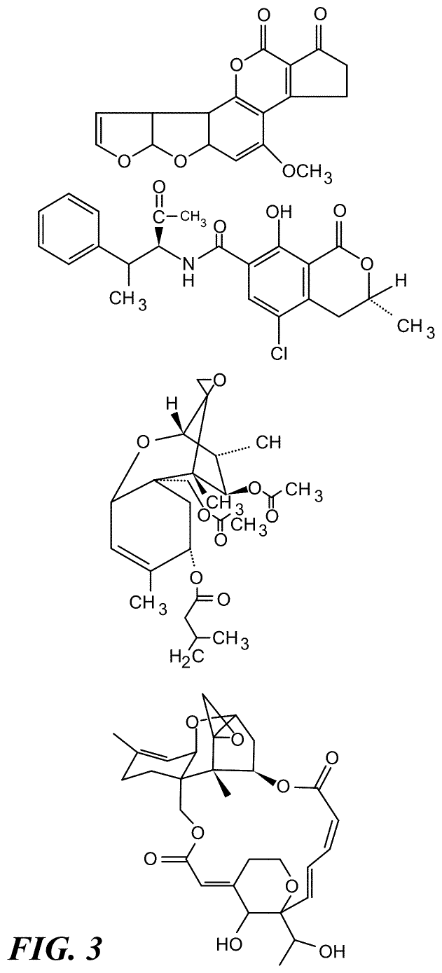

A small biological molecule for detection and/or quantification can be a primary or a secondary metabolite produced by an animal or a plant. It can be a metabolite produced by a microorganism or a fungus. Mycotoxins are low-molecular-weight natural products (i.e., small molecules) produced as secondary metabolites by filamentous fungi. These metabolites constitute a toxigenically and chemically heterogeneous assemblage of molecules that are grouped together only for their ability to cause disease and death in plants and animals (Bennett, J W 1987). Hundreds of mycotoxins are known, some of which are of primary concern to humans because of the effects they produce through either direct exposure to them in indoor environments (Hendry K M et al., 1993) or indirectly through food contaminated with them. Structures of four such mycotoxins, aflatoxin B1, ochratoxin A, T-2 toxin, and satratoxin H, are shown in FIG. 3. Aflatoxins of four major types, B1, B2, G1, and G2, are known, among which B1 is the most common. Acute aflatoxicosis can result in death and chronic aflatoxicosis is associated with cancer, immune suppression, and other “slow” pathological conditions (Hsieh, D, 1988). Ochratoxin A is a nephrotoxin, a liver toxin, an immune suppressant, a potent teratogen, and a carcinogen (Beardall and Miller, 1994). It is produced by multiple species of Aspergillus. Aspergillus niger is used widely in the production of enzymes and citric acid for human consumption. As such, it is important to ensure that industrial strains do not produce this toxin (Téren, J et al., 1996). T-2 toxin is part of a class of molecules known as trichothecenes and is produced by a number of fungal genera, including Fusarium, Myrothecium, Phomopsis, Stachybotrys, Trichoderma, and Trichothecium (Scott, P M, 1989). T-2 toxin has been detected in the dust from office ventilation systems (Smoragiewicz, W B, et al., 1993). Satratoxin H also is a trichothecene. It is produced by Stachybotrys chartarum, also known as black mold. It causes the disease Stachybotryotoxicosis, first described as an equine disease of high mortality associated with moldy straw and hay. Stachybotrys grows well on all sorts of wet building materials with high cellulose content, for example, water-damaged gypsum board, ceiling tiles, wood fiber boards, and even dust-lined air conditioning ducts (Nikulin, M et al., 1994). No method for detecting Stachybotrys mycotoxins in known although methods evaluating the presence of Stachybotrys chartarum by PCR are known (Vesper, S J et al., 2000).

While an immune response requires the C1q protein to bind to two or more antibody molecules to initiate the next step in the complement response, binding to a single antibody molecule is sufficient for the operation of the present sensor. The terms “C1q protein”, “C1q polypeptide”, and “C1q complex” are used interchangeably herein to refer to the hexameric complex composed of C1q proteins A, B, and C, which forms a functional unit for binding selectively to antigen-bound antibody, but does not bind to antibody that is not bound to antigen. Both native and recombinant C1q can be used, and the source of the C1q or its sequence (for recombinant C1q) can be human C1q or a C1q from any mammalian species. Typically, the C1q comples is devoid of other proteins, such as complement proteins that lead to the initiation or execution of the complement cascade. Native human C1q is commercially available, and recombinant human C1q can be prepared according to published methods. See, for example, Bally, et al., 2013. Isolation of murine C1q protein has been reported (McManus L M and Nakane, P K, 1980). While the species of C1q can be matched to the species of immunoglobulin, cross-species interactions are also possible. For example, human C1q can recognize antigen-bound murine antibodies (Seino J et al., 1993). Chimeric antibodies having murine variable and human constant regions, as well as fully human antibodies, e.g., those described in U.S. Pat. No. 5,939,598, may also be used in conjunction with both human and murine C1q proteins. Fully murine antibodies may also be used, or antibodies of another mammalian species.

The present technology contemplates using a fragment of C1q instead of the full-length C1q for binding to antigen-bound antibody. The fragment includes the region of C1q responsible for binding to the antibody. Also contemplated are variants of the fragment that retain binding to the antigen-bound antibody. C1q fragments can be prepared using the procedure described in Gaboriaud, et al. (2003). C1q is a 460-kDa protein made of six heterotrimeric collagen-like triple helices. The helices associate in their N-terminal half to form a “stalk,” diverging thereafter to form individual “stems”, each terminating in a C-terminal heterotrimeric globular domain. These heterotrimeric globular domains or heads recognize most of the C1q complex ligands. The C1q fragment described in Gaboriaud, et al. is made of the C-terminal heterotrimeric globular domain and was generated by digesting C1q with collagenase. The amino acid sequences of individual subunits, i.e., human C1q-A (SEQ ID NO:1), C1q-B (SEQ ID NO:2), and C1q-C(SEQ ID NO:3), produced as a result of the digestion, are shown below:

| | |
|---|---|
| clq_a | QPRPAFSAIRRN--PPMGGNVVIFDTVITNQEEPYQNHSGRFVCTVPGYYYFTFQVLSQ-WEICLSIVSSS |
| clq_b | TQKIAFSATRTINVPLRRDQTIRFDHVITNMNNNYEPRSGKFTCKVPGLYYFTYHASSR-GNLCVNLMRG- |
| cla_c | KFQSVFTVTRQTHQPPAPNSLIRFNAVLTNPQGDYDTSTGKFTCKVPGLYYFVYHASHT-ANLCVLLYRS- |
| clq_a | RGQVRRSLGFCDTTNKGLFQVVSGGMVLQLQQGDQVWVEKD--PKKGHIYQGSEADSVFSGFLIFPS A |
| clq_b | RERAQKVVTFCDYAYN-TFQVTTGGMVLKLEQGENVFLQAT--DKNSLLGMEG-ANSIFSGFLLFPD MEA |
| clq_c | -G--VKVVTFCGHTSK-TNQVNSGGVLLRLQVGEEVWLAMN--DYYDMVGIQG-SDSVFSGFLLFPD |

Variants of full length C1q also can be used in place of native mammalian C1q. C1q variants of either full length C1q or of a C1q fragment preferably have at least 95% identity with the amino acid sequence of the respective native C1q or fragment thereof from which the variant was derived. In alternative embodiments, the level of sequence identity is at least 80%, at least 85%, at least 90%, at least 97%, at least 98%, or at least 99% compared to the amino acid sequence of the respective native C1q or fragment thereof from which the variant was derived. In yet other embodiments, the variant differs from the native sequence only by one or more, 2 or more, 5 or more, 10 or more, or 20 or more conservative amino acid substitutions. Further, a C1q variant can include variants of one, two, or all three of the constituent chains (A, B, and C), with non-variant portions made up by native, naturally occurring sequences. The percent identify of a C1q variant refers to the identity of the total complex of 18 polypeptide chains with respect to a native total complex of 18 chains.

Voltammetric detection of an analyte can be performed in complex fluid media, such as a body fluid sample, tissue extract, or cell culture medium, without prior separation or purification of the analyte from the mixture (Webster, T A et al., 2014; Webster, T A et al., 2015; Sismaet, H J et al., 2016b). Also, voltammetric detection can be performed in complex samples such as soil extracts and seawater (Cash, K J et al., 2009a; Cash, K J et al., 2009b; Patterson, A S et al., 2013a; Patterson, et al. 2013b). This is accomplished by utilizing redox-active molecules that have half-wave potentials in the window of −0.4 to 0.0 volts, with a Ag/AgCl electrode being used as reference. In voltammetric detection (unlike capacitive detection), components other than the molecule of interest do not cause significant interference with current output, allowing measurements to be made in a variety of samples having different chemical environments and sources.

Multiple electrochemical sensors of the kind described above may be used in a single device (e.g., in the form of an array) for simultaneous detection of several different analytes. Such a device may be used, for example, to simultaneously detect multiple toxins or to distinguish among multiple pathogens using the same redox functionalized C1q.

### EXAMPLES

### Example 1. Optimizing C1q-Antibody Binding Using SPRi and QCM

Binding of C1q to antibodies bound to antigens is demonstrated by a surface plasmon resonance (SPR) imaging system (SPRi-Lab+ system, Horiba Scientific) and a quartz crystal microbalance (QCM-D, 3T Analytik) using biotin as the antigen. Sensing by both SPR and QCM has been used to detect protein binding events (Abadian P N, et al., 2014; Abadian and Goluch, 2015; Sismaet, H J et al., 2016a). Also, SPR has previously been used to study binding of C1q to antibodies (He, J et al., 2014). C1q is available from Abcam (ab96363).

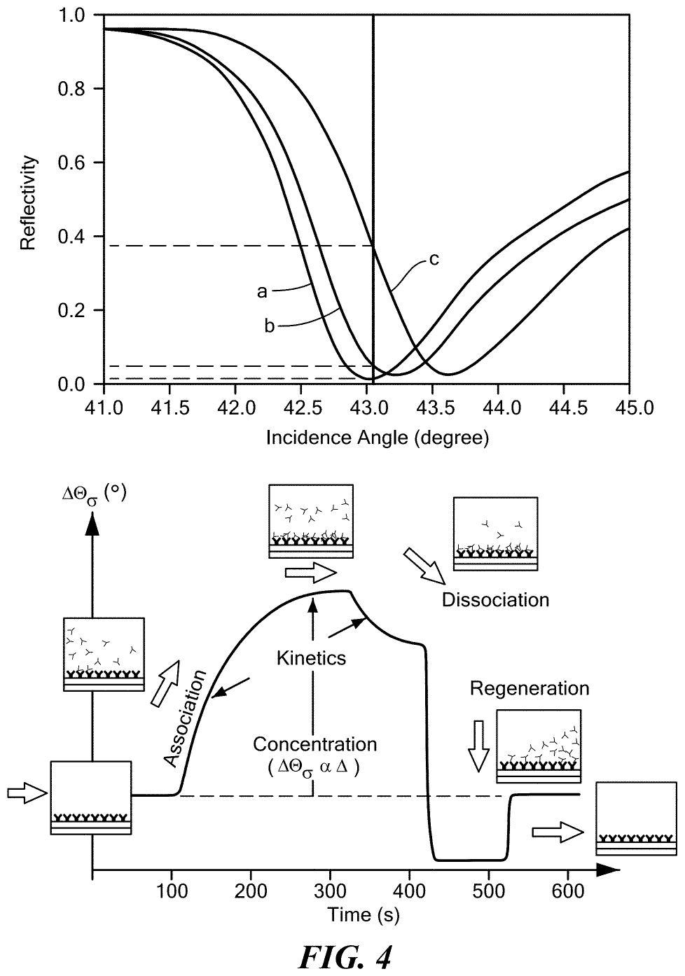

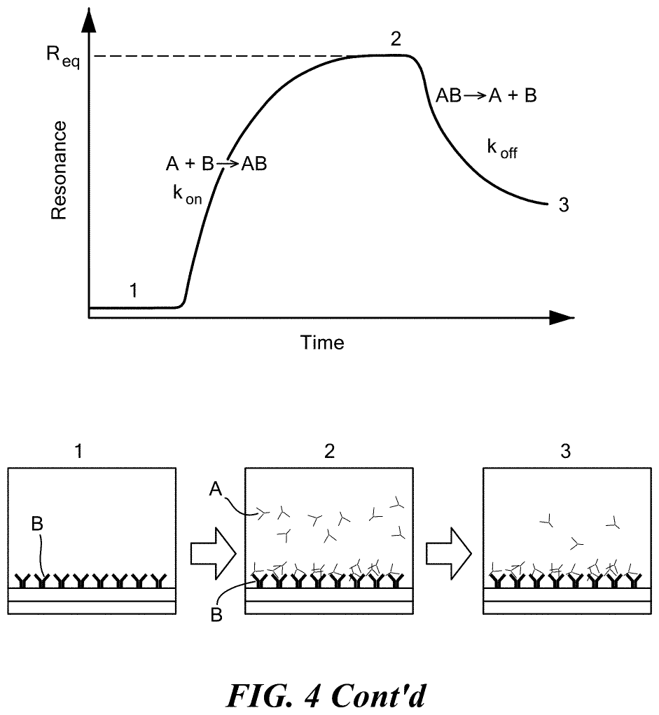

The general approach for determining binding kinetics using SPR is demonstrated in FIG. 4. As part of optimization, parameters related to successful binding of C1q to antibodies immobilized on the gold sensor surface is determined. Direct attachment of antibody to the gold on SPR and QCM sensor surfaces can result in too much steric hindrance for C1q to bind the antibody. Hence, a bifunctional linker with a carbon chain spacer is preferably used to allow the antibodies to be located away from the gold surface and in solution. Attachment of carboxyl groups on the linker to lysine residues on the antibody, or vice versa, can be used. However, the location of the attachment on the antibody cannot be controlled using this approach. The linker also can be attached to the sulfur atoms of disulfide bonds that hold the two arms of the antibody together. The disulfide bonds are broken with a reducing agent. Multiple options are available for attaching the linker to the gold sensor surface. In one approach, the gold on the sensor surface is passivated with a lysine terminated group. The bifunctional linker used has a carboxyl group on one side and a peptide bond is formed using standard EDC/NHS chemistry. The other end of the spacer is attached to the antibody under oxidizing conditions. The length of the carbon chain of the spacer can be varied, and a suitable length selected based on binding data. In addition, different sensor surface passivation strategies can be used to prevent the antibodies from adhering to the surface.

After the antibodies are immobilized, the functionalized sensors are tested using SPRi and QCM instruments. First, a solution containing antigen (e.g., biotin as test analyte) is flowed past the sensor surface, during which antigen binds to the antibodies. Then, the C1q is introduced and the response recorded. The surface density of antibodies is varied and the experiment repeated to see how C1q binding levels change.

### Example 2: Attachment of a Tethered Redox Active Molecule to C1q

C1q is not redox active on its own. Therefore, to detect binding events based on complex formation between an antibody and C1q using square wave voltammetry, a redox-active molecule is utilized. C1q is modified with a bifunctional linker. To the modified C1q is attached methylene blue or another redox-active molecule. Methylene blue transfers electrons to the electrode at −0.3 V, with a Ag/AgCl electrode used as reference. The redox molecule must be free to move in order to maximize the current produced by maximizing contact between the redox molecule and the electrode surface. Therefore, the redox active molecule is tethered to C1q using a tether having a suitable length to optimize contact of the redox active molecule with the electrode. Bifunctional linkers having different carbon chain lengths (e.g., 5-30 carbons atoms) are tested as tethers and a suitable length is selected. In addition, bifunctional linkers having single stranded DNA as the spacer instead of a carbon chain also can be tested. Excessively long spacers are expected to lower the measured current per bound C1q molecule because the redox molecule is less likely to touch the electrode surface. An SPRi system having an open flow cell is used, which allows coupling electrochemical measurements with the SPR using a potentiostat. This permits validation of binding events detected through electrochemical measurements.

### Example 3: Multiplexed Sensing of Mycotoxins Using C1q-Antibody Electrochemical Sensing

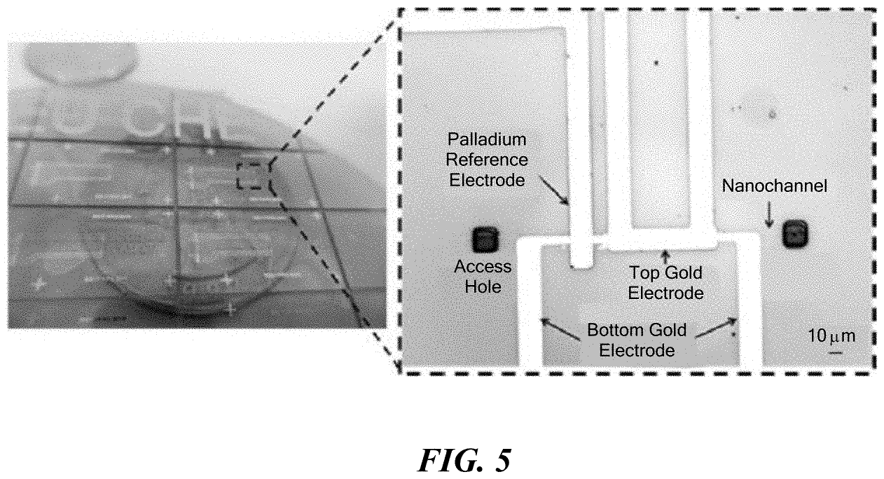

The C1q-antibody-based electrochemical sensor of Example 2 is utilized to develop a multiplexed sensor or device capable of detecting four mycotoxins. These mycotoxins are chosen because they affect food supply and human health. Antibodies against the mycotoxins are obtained from a commercial source (Abcam). Alternatively, novel antibodies against these antigens are produced. A multielectrode device for electrochemical sensing is fabricated, with each electrode functionalized with a different antibody, so as to distinguish which antigen is present in the sample. An example of a microfabricated nanofluidic device is shown in FIG. 5 (Webster and Goluch 2012, Webster, T A et al., 2014). The mycotoxins are dissolved in salt buffered solution, such as 0.2 mM NaCl PBS at pH 7. The sensitivity and specificity of each of the individual sensors is determined.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present technology has been described in conjunction with certain preferred embodiments, one of ordinary skill, after reading the foregoing specification, will be able to effect various changes, substitutions of equivalents, and other alterations to the compositions and methods set forth herein.

### REFERENCES

Abadian, P. N. and E. D. Goluch (2015). “Surface plasmon resonance imaging (SPRi) for multiplexed evaluation of bacterial adhesion onto surface coatings.” Analytical Methods 7(1): 115-122.
Abadian, P. N., N. Tandogan, J. J. Jamieson and E. D. Goluch (2014). “Using surface plasmon resonance imaging to study bacterial biofilmsa).” Biomicrofluidics 8(2): 021804.
Bally I, Ancelet S, Moriscot C, Gonnet F, Mantovani A, Daniel R, Schoehn G, Arlaud G J, Thielens N M (2013), Expression of recombinant human complement C1q allows identification of the C1r/C1s-binding sites, Proc Natl Acad Sci USA. 2013 May 21; 110(21):8650-5
Beardall, J. M. and J. D. Miller (1994). Disease in humans with mycotoxins as possible causes. St. Paul, Eagan Press.
Bennett, J. W. and M. Klich (2003). “Mycotoxins.” Clinical Microbiology Reviews 16(3): 497¬516.
Cash, K. J., F. Ricci and K. W. Plaxco (2009a). “An Electrochemical Sensor for the Detection of Protein-Small Molecule Interactions Directly in Serum and Other Complex Matrices,” Journal of the American Chemical Society 131(20): 6955-6957.
Cash, K. J., F. Ricci and K. W. Plaxco (2009b). “A general electrochemical method for label-free screening of protein-small molecule interactions.” Chemical Communications (41): 6222-6224.
Gaboriaud, C., et al. (2003), “The Crystal Structure of the Globular Head of Complement Protein C1q Provides a Basis for Its Versatile Recognition Properties,” J. Biol. Chem. 278, 46974-46982
He, J., H. Lai, M. Engle, S. Gorlatov, C. Gruber, H. Steinkellner, M. S. Diamond and Q. Chen (2014). “Generation and Analysis of Novel Plant-Derived Antibody-Based Therapeutic Molecules against West Nile Virus.” PLoS ONE 9(3): e93541.
Hendry, K. M. and E. C. Cole (1993). “A review of mycotoxins in indoor air.” Journal of Toxicology and Environmental Health 38(2): 183-198.
Hsieh, D. (1988). Potential human health hazards of mycotoxins. Amsterdam, Elsevier.
Janeway, C. A., P. Travers, M. Walport and M. J. Shlomchik (2001). Immunobiology: The Immune System in Health and Disease. 5th edition. New York, Garland Science.
Moldave, K. (2003). Progress in Nucleic Acid Research and Molecular Biology. Netherlands, Academic Press. National Academy of Engineering, Changing the Conversation: Messages for Improving Public Understanding of Engineering. 2008: The National Academies Press.
McManus L M, Nakane P K, Effect of steryl glycosides on the phase transition of dipalmitoyl lecithin, Plant Physiol. 1980 January; 65(1):78-80.
Nikulin, M., A. L. Pasanen, S. Berg and E. L. Hintikka (1994). “Stachybotrys atra Growth and Toxin Production in Some Building Materials and Fodder under Different Relative Humidities.” Appl Environ Microbiol 60(9): 3421-3424.
Patterson, A. S., D. M. Heithoff, B. S. Ferguson, H. T. Soh, M. J. Mahan and K. W. Plaxco (2013a). “Microfluidic Chip-Based Detection and Intraspecies Strain Discrimination of Salmonella Serovars Derived from Whole Blood of Septic Mice.” Applied and Environmental Microbiology 79(7): 2302-2311.
Patterson, A. S., K. Hsieh, H. T. Soh and K. W. Plaxco (2013b). “Electrochemical real-time nucleic acid amplification: towards point-of-care quantification of pathogens.” Trends in Biotechnology 31(12): 704-712.
Scott, P. M. (1989). The natural occurrence of trichothecenes. Boca Raton, CRC Press.
Sismaet, H. J., T. A. Webster, and E. D. Goluch, “Up-regulating Pyocyanin Production by Amino Acid Addition for Early Identification of Pseudomonas aeruginosa.” Analyst, In Press (2014).
Seino J, Eveleigh P, Warnaar S, van Haarlem L J, van Es L A, Daha M R, Activation of human complement by mouse and mouse/human chimeric monoclonal antibodies, Clin Exp Immunol. 1993 November; 94(2):291-6.
Sismaet, H. J., P. N. Abadian and E. D. Goluch (2016a). Applications of a Quartz Crystal Microbalance for Monitoring Bacterial Biofilm Growth and Removal. PittCon 2016, New Orleans, L A.
Sismaet, H. J., A. Banerjee, S. McNish, Y. Choi, M. Torralba, S. Lucas, A. Chan, V. K. Shanmugam and E. D. Goluch (2016b). “Electrochemical detection of Pseudomonas in wound exudate samples from patients with chronic wounds.” Wound Repair and Regeneration 24(2): 366-372.
Smoragiewicz, W., B. Cossette, A. Boutard and K. Krzystyniak (1993). “Trichothecene mycotoxins in the dust of ventilation systems in office buildings.” Int Arch Occup Environ Health 65(2): 113-117.
Téren, J., J. Varga, Z. Hamari, E. Rinyu and F. Kevei (1996). “Immunochemical detection of ochratoxin A in black Aspergillus strains.” Mycopathologia 134(3): 171-176.
Vesper, S., D. G. Dearborn, I. Yike, T. Allan, J. Sobolewski, S. F. Hinkley, B. B. Jarvis and R. A. Haugland (2000). “Evaluation of Stachybotrys chartarum in the house of an infant with pulmonary hemorrhage: quantitative assessment before, during, and after remediation.” J Urban Health 77(1): 68-85.
Webster, T. A., H. J. Sismaet, D. R. Hunt, and E. D. Goluch. “Monitoring Pseudomonas aeruginosa in Culture Plates using Embedded Electrochemical Sensors,” in 40th Northeast Bioengineering Conference (NEBEC 2014). 2014. Boston, Mass. USA. IEEE.
Webster, T. A., H. J. Sismaet, and E. D. Goluch. “Electrochemical Monitoring of Pseudomonas aeruginosa Biofilms in Microfluidic Channels,” in The 18th International Conference on Miniaturized Systems for Chemistry and Life Sciences (microTAS 2014). 2014. San Antonio, Tex., USA.
Webster, T. A. and E. D. Goluch (2012). “Electrochemical detection of pyocyanin in nanochannels with integrated palladium hydride reference electrodes.” Lab on a Chip 12(24): 5195-5201.
Webster, T. A., H. J. Sismaet, I. p. J. Chan and E. D. Goluch (2015). “Electrochemically monitoring the antibiotic susceptibility of Pseudomonas aeruginosa biofilms.” Analyst 140(21): 7195-7201.
Webster, T. A., H. J. Sismaet, J. L. Conte, I. p. J. Chan and E. D. Goluch (2014). “Electrochemical detection of Pseudomonas aeruginosa in human fluid samples via pyocyanin.” Biosensors and Bioelectronics 60: 265-270.

1. A method for detecting an analyte, the method comprising the steps of:
(a) providing
(i) a device comprising
a working electrode having a detection surface;
an antibody covalently linked to the detection surface, wherein the antibody is capable of specifically binding to the analyte;
a reference electrode; and
circuitry for applying a voltage between the working electrode and the reference electrode and measuring a current produced by a redox reaction at the detection surface;
(ii) a sample suspected of containing the analyte; and
(iii) a C1q polypeptide, or a fragment or variant thereof, covalently linked to a redox active molecule by a tether, wherein the C1q polypeptide, or fragment or variant thereof, is capable of binding to a complex formed by binding of the analyte to the antibody covalently linked to the detection surface of the device;
(b) contacting the sample with the detection surface of the device, whereby the antibody covalently linked to the detection surface binds to analyte in the sample to form an antibody-analyte complex;
(c) contacting the C1q polypeptide, or fragment or variant thereof, with the antibody-analyte complex, whereby the redox active molecule contacts the detection surface; and
(d) applying a voltage between the working electrode and the reference electrode of the device, whereby an electron transfer reaction of the redox active molecule is detected by the device as a current between the working electrode and the reference electrode.

2. The method of claim 1, wherein current is measured in step (d) in response to one or more square wave potentials.

3. The method of claim 1, further comprising determining a concentration of the analyte by applying a previously determined correlation between the current measured in step (d) and concentration of the analyte.

4. The method of claim 1, wherein the device further comprises one or more additional working electrodes, each additional working electrode comprising a detection surface to which is bound a unique antibody that specifically binds to a unique analyte, and two or more analytes are detected.

5. The method of claim 1, wherein the device comprises a C1q polypeptide variant, wherein the variant has at least 95% identity with the amino acid sequence of a mammalian C1q polypeptide.

6. The method of claim 1, wherein the device further comprises one or more additional antibodies covalently linked to said detection surface or to one or more additional detection surfaces on one or more additional electrodes, wherein each of said one or more additional antibodies is capable of specifically binding to a unique additional analyte.

7. The method of claim 6, wherein the device comprises two or more separate electrodes, each electrode comprising a detection surface to which is bound a unique antibody that specifically binds to a unique analyte.

8. The method of claim 1, wherein the redox active molecule has a half-wave potential in the range from about −0.4 volts to about 0.0 volts with respect to a Ag/AgCl reference electrode.

9. The method of claim 8, wherein the redox active molecule is methylene blue.

10. The method of claim 1, wherein the analyte has a molecular weight of less than 10,000 Daltons.

11. The method of claim 10, wherein the analyte has a molecular weight of less than 1,000 Daltons.

12. The method of claim 1, wherein the analyte is a biomolecule.

13. The method of claim 12, wherein the biomolecule is a cytokine, a hormone, a peptide, a polypeptide, a nucleic acid, a sugar, or a polysaccharide.

14. The method of claim 12, wherein the biomolecule is from a pathogen.

15. The method of claim 12, wherein the biomolecule has a molecular weight of 10,000 Daltons or greater.

16. The method of claim 1, wherein the analyte is a bacterial toxin or a mycotoxin.

17. The method of claim 1, wherein the analyte is a pharmaceutical agent.
